(19) United States
(12) Patent Application Publication
(10) Pub. No.: US 2021/0353980 A1
(43) Pub. Date: Nov. 18, 2021

(54) MULTI-FLAP VALVE FOR A RESPIRATORY DEVICE

(71) Applicant: INNOSPARKS PTE LTD, Singapore (SG)

(72) Inventors: Er Ong Wern, Singapore (SG); Gareth Tang Ee Ho, Singapore (SG); Jerome Wei Liang LEE, Singapore (SG); Meng Wai MUN, Singapore (SG); Mikail LO, Singapore (SG)

(21) Appl. No.: 16/331,172

(22) PCT Filed: May 2, 2018
(86) PCT No.: PCT/SG2018/050212

§ 371 Date: Nov. 7, 2019

(51) Int. Cl. A62B 18/10; F16K 15/14; A62B 18/02

(52) U.S. Cl. CPC A62B 18/10; A62B 18/025; F16K 15/147

(57) ABSTRACT

Embodiments of the invention include a unidirectional exhalation valve for a respiratory device such as a facemask. The valve opens to vent air out of the interior of the facemask and closes to prevent the inflow of ambient air. The valve is comprised of a valve body, a valve cover and a membrane. The membrane is secured to the valve body around its perimeter by the valve cover. The membrane is comprised of two or more flaps that flex toward a central region, in a direction away from the valve body, to open the unidirectional valve.

### FIELD OF THE INVENTION

The invention relates to respiratory devices, and more specifically, to a unidirectional valve for a respiratory device such as a facemask for releasing exhaled air and blocking inflow of ambient air.

### BACKGROUND

Face masks that cover the nose and mouth of a wearer are often worn to filter airborne particles from ambient air. A typical respiratory facemask includes a filtering material that forms a seal with the face by enclosing the nose and mouth. A common feature of such facemasks is a unidirectional exhalation valve. The valve allows exhaled air to be purged from the mask body with less resistance to air flow than the filter material of the mask. This improves the comfort and effectiveness of the mask by promoting the release of expired air. Because it is unidirectional, inhaled air is directed through the filtering portion of the mask.

An exhalation valve typically includes an opening sealed by a flexible membrane. The flexible membrane is attached to a base at one edge. The membrane forms a seal over the opening with neutral or negative pressure in the mask body. The membrane flexes open with positive pressure to open the valve. The free edges release the seal and allow air to pass through the valve. With this design, air passes in one direction (i.e. outward) through the exhalation valve.

Unidirectional valves inherently cause resistance to the flow of air. Air pressure is needed to open the valve by flexing the membrane. Air is then diverted through a path around the membrane. Exhaled air may not be adequately purged from the facemask due to this resistance. The problem is more apparent with a larger internal respirator space (i.e. more dead space). Moreover, with shallow breathing, the positive pressure may be inadequate to open the valve by flexing the membrane. This reduces the effectiveness of the respirator. The wearer may experience a higher temperature, humidity and carbon dioxide level in the mask. Recent efforts have focused on improving the seal and reducing the resistance to the flow of air in exhalation valves.

For example, U.S. Pat. No. 4,414,973 describes a facemask with a valve that has a round membrane secured at its center. The valve opens during exhalation when an edge of the membrane flexes to allow air to pass through the valve. The membrane includes flexible staggered ribs that flex with a pressure differential. While the design may offer some improvements, exhaled air must follow a diverted path. As with conventional designs, this creates resistance to the outward flow of exhaled air.

Similarly, U.S. Patent 2016/0074682 describes a round membrane that is secured to a base at a center point. The membrane has a “butterfly” shape intended to increase its flexibility and reduce the resistance to air flow during exhalation. However, it functions as a conventional valves and exhaled air must also follow a diverted path.

U.S. Pat. No. 4,934,362 describes a rectangular shaped membrane that is secured at its center and curls up on both ends with positive pressure inside the mask body. Circular flexible flaps on the membrane allow portions of it to shift when the user exhales. As with conventional designs, exhaled air must force the membrane open and then follow a path around the membrane. This creates resistance air flow and limits the amount of exhaled air that is purged.

In other designs of unidirectional valves, a flexible membrane is mounted off-center with respect to an opening. See, for example, U.S. Pat. Nos. 5,325,892 and 8,365,771. This may help lower the exhalation pressure necessary to open the valve. However, as with other conventional valves, air must follow a diverted path to exit the respirator which creates resistance.

While these designs may offer improvements, they inherently create resistance to the outward flow of exhaled air. For this reason, exhaled air may not be effectively purged from the mask. Further, exhaled air is pushed through the filter material which absorbs heat and moisture. This can lead to discomfort, especially if the mask is worn for extended periods of time.

Accordingly, there is a need for an improved unidirectional exhalation valve for a respiratory device. It should maintain a firm seal to block air from entering during inhalation and have a low resistance to the flow of air out of the mask during exhalation.

### SUMMARY OF THE INVENTION

The following summary is provided to facilitate an understanding of some of the innovative features unique to the disclosed embodiment and is not intended to be a full description. A full appreciation of the various aspects of the embodiments disclosed herein can be gained by taking into consideration the entire specification, claims, drawings, and abstract as a whole.

Embodiments include a unidirectional valve for a respiratory device such as a facemask. The unidirectional valve includes a valve body, a valve cover and a membrane. The membrane is secured around a perimeter region to the valve body by the valve cover. The membrane is comprised of a flexible material with two or more flaps that move independently such that each flap flexes at a hinge region. The membrane opens at a central region when the two or more flaps flex in a direction away from the valve body. The valve is opened from positive pressure within a body region of respiratory device.

The membrane forms a seal with the valve body at a sealing surface. The sealing surface can have a substantially flat shape or a curved shape. The membrane is secured to the valve body at a mounting surface. The mounting surface can have a substantially flat shape or a curved shape. The mounting surface can be comprised of one or more points where the membrane is affixed to the valve body and/or the valve cover

The flaps of the membrane can be formed by cuts in the membrane and can flex at or near hinge regions. The flaps can be formed from curved vertices in the membrane. In the alternative, the membrane can be comprised of one or more panels.

Embodiments also include a membrane for a unidirectional valve. The membrane is comprised of a flexible material with two or more flaps that move independently such that each flap flexes at a hinge region. The membrane is secured around a perimeter region to a valve body of the unidirectional valve. The membrane opens when the flaps flex in a direction away from the valve body.

### INTRODUCTION

In a first embodiment, there is provided a unidirectional valve for a respiratory device such as a face mask.

In a second embodiment, there is provided a unidirectional valve comprised of a valve cover, a membrane and a valve body.

In a third embodiment, there is provided a membrane for a unidirectional valve with one or more flexible flaps that close the valve when a wearer inhales and open the valve when a wearer exhales.

In a fourth embodiment, there is provided a membrane for a unidirectional valve with one or more flexible flaps that open the valve when a wearer exhales to allow air flow with minimal resistance.

In a fifth embodiment, there is provided a unidirectional valve with a curved sealing surface where the membrane forms a seal with the valve body.

In a sixth embodiment, there is provided a unidirectional valve with a curved mounting surface where the membrane is secured to the valve body.

In a seventh embodiment, there is provided a membrane comprised of multiple flexible flaps that flex at hinge regions to open the membrane.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the disclosure is not limited to specific methods and instrumentalities disclosed herein. Moreover, those in the art will understand that the drawings are not to scale. Wherever possible, like elements have been indicated by identical numbers.

FIG. 1 depicts a respiratory facemask with an exhalation valve, according to one embodiment.

FIG. 2 depicts a cross-sectional view of an exhalation valve, according to one embodiment.

FIG. 3A depicts an exploded view of the components of an exhalation valve, according to one embodiment.

FIG. 3B depicts a top view of an exhalation valve membrane with four symmetrical flaps, according to one embodiment.

FIG. 4 depicts a top view of an exhalation valve membrane with areas of contact between the membrane and the valve body, according to one embodiment.

FIG. 5A depicts a membrane with four symmetrical flaps, according to one embodiment.

FIG. 5B depicts a membrane with a spacing (i.e. gaps) between the flaps, according to one embodiment.

FIG. 5C depicts a membrane with flaps with curved vertices, according to one embodiment.

FIG. 5D depicts a membrane with four curved flaps and spacing between the flaps, according to one embodiment.

FIG. 6 depicts a cross-sectional view of an exhalation valve with a curved sealing surface, according to one embodiment.

FIG. 7A depicts a top view of an exhalation valve when the membrane is closed, according to one embodiment.

FIG. 7B depicts a bottom view of an exhalation valve when the membrane is closed, according to one embodiment.

FIG. 7C depicts a perspective view of an exhalation valve when the membrane is closed, according to one embodiment.

FIG. 7D depicts a cross-sectional view of an exhalation valve when the membrane is closed, according to one embodiment.

FIG. 8 depicts a cross-sectional view of an exhalation valve and the forces acting on the valve during inhalation, according to one embodiment.

FIG. 9A depicts a top view of exhalation valve when the membrane is open, according to one embodiment.

FIG. 9B depicts a bottom view of an exhalation valve when the membrane is open, according to one embodiment.

FIG. 9C depicts a perspective view of an exhalation valve when the membrane is open, according to one embodiment.

FIG. 9D depicts a cross-sectional view of an exhalation valve when the membrane is open, according to one embodiment.

FIG. 10 depicts a cross-sectional view of an exhalation valve and the path of airflow during exhalation, according to one embodiment.

FIG. 11A depicts a membrane with two rectangular flaps, according to one embodiment.

FIG. 11B depicts a membrane with two curved flaps, according to one embodiment.

FIG. 11C depicts a membrane with two curved flaps with longer hinge regions, according to one embodiment.

FIG. 11D depicts a membrane with two elliptical flaps, according to one embodiment.

FIG. 11E depicts a membrane with two triangular flaps, according to one embodiment.

FIG. 11F depicts a membrane with three triangular flaps, according to one embodiment.

FIG. 11G depicts a membrane with four symmetrical flaps, according to one embodiment.

FIG. 11H depicts a membrane with five triangular flaps, according to one embodiment.

FIG. 11I depicts a membrane with six triangular flaps, according to one embodiment.

FIG. 11J depicts a membrane comprised of four panels of equal size and shape, according to one embodiment.

FIG. 11K depicts a membrane with non-identical flaps, according to one embodiment.

FIG. 12A depicts a rectangular shaped membrane with two rectangular flaps, according to one embodiment.

FIG. 12B depicts a rectangular shaped membrane with curved flaps, according to one embodiment.

### DETAILED DESCRIPTION

The particular values and configurations discussed in these non-limiting examples can be varied and are cited merely to illustrate at least one embodiment and are not intended to limit the scope thereof.

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can, in certain instances, be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

The term “mechanical filter” refers to a respirator that retains particulate matter such as dust primarily by interception and impaction with fibers of the respirator.

The term “membrane” or “diaphragm” refers to a thin, flexible sheet (e.g. rubber, silicone or plastic) that forms an airtight seal as a component of a valve.

The term “N95 respirator” refers to a respiratory protective device designed to achieve a close facial fit and efficient filtration of airborne particles, such that the respirator blocks at least 95% of non-oil air particulates.

Other technical terms used herein have their ordinary meaning in the art that they are used, as exemplified by a variety of technical dictionaries.

### DESCRIPTION OF PREFERRED EMBODIMENTS

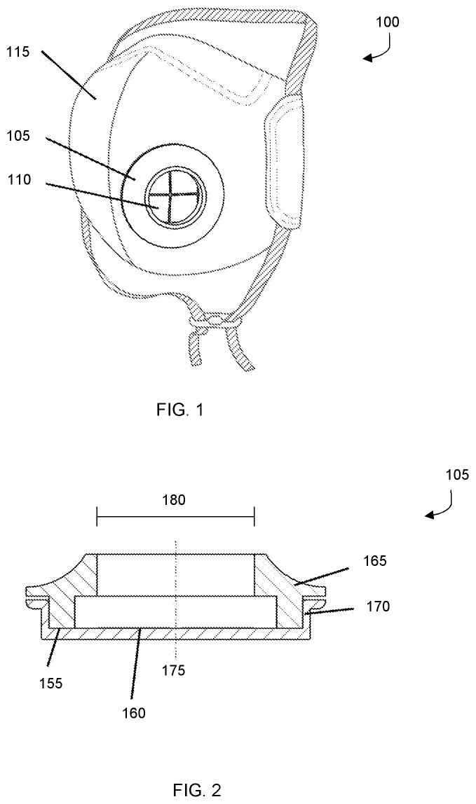

FIG. 1 depicts a mask 100 that includes a unidirectional valve 105 according to one embodiment. During normal use, the mask body 115 covers the mouth and nose of a wearer. Incoming air (i.e. ambient air) is filtered through the mask body 115 to protect the wearer from inhaling dust, germs or other particulate matter in the air that can be potentially harmful. The mask can be made for multi or single use (i.e. disposable). The mask body 115 can be comprised of an air-filtering porous (i.e. air purifying) material and an exhalation valve 105. In this design, the unidirectional valve 105 is mounted off center.

When the wearer of a mask inhales, the exhalation valve remains closed.

Ambient air is filtered as it passes through the filtering material of the mask body. A tight membrane seal against the valve body ensures that air entering the mask body is filtered. A loose seal could allow undesirable airborne particulates to enter the mask body.

When the wearer exhales, the exhalation valve opens and air exits the mask primarily through the valve as it is a preferred path of least resistance. It will be appreciated that a small amount of exhaled air may pass through the filter material of the mask itself, but with much more resistance in comparison to the path through the valve. During exhalation the air flows through the valve with less resistance than during inhalation due to the unobstructed valve opening. This one-way valve action promotes the removal of expired air from within the mask to improve the comfort for the wearer, while also ensuring that inhaled air is filtered.

The unidirectional valve is comprised of a membrane 110 that responds to air pressure directed against it either externally to the mask body or internally to the mask body. During inhalation, pressure inside the mask body decreases such that the membrane 110 remains in a sealed position and the valve remains closed. This prevents air from flowing through the valve such that inhaled air is drawn through the filter material of the mask. During exhalation, pressure inside the mask increases such that the valve opens and offers a preferred path of least resistance for the exhaled air to flow outward through the valve and into the ambient environment.

In conventional exhalation valves, the membrane is typically secured to the valve body at one peripheral edge or portion with the remaining peripheral edges or portions being free (i.e. non-secured or non-attached); or the membrane is secured or pinned to the valve body at its center with the entire peripheral edges being free. In both of these conventional valve designs, the valve functions to open around the peripheral edges and free portions of the membrane to allow air to flow through. These conventional valve designs are closed when the free portions of the membrane are tightly seated (i.e. attached or secured) against the valve body around the peripheral edges and free portions of the membrane.

Conventional exhalation valves that open around at least a peripheral edge of the membrane require a reasonably high amount of force and air pressure to move the membrane and open the valve so that air can flow through. Accordingly, in order to facilitate the movement of the conventional membrane, the flow of exhaled air directed at the valve is obstructed and the must be re-directed to at least a peripheral edge of the membrane resulting in increased air flow resistance.

Embodiments of the invention described herein include a unidirectional valve with less resistance to exhaled air flow than conventional valves. In particular, instead of securing the membrane at an edge or its center, the membrane is secured around its perimeter without the need to secure or attach the membrane at its center point or middle portion. The membrane is divided into at least two flaps or panels that flex along hinge regions to open the valve in a central region or orifice in response to the appropriate air pressure and exhaled air flow.

FIG. 2 depicts a cross-sectional view of a valve 105 according to one embodiment. A valve cover 165 secures a membrane to a substantially flat sealing surface 160 on the valve body 170. The area where the membrane is affixed or secured to valve body is known as a mounting surface 155. A central point 175 and central region 180 are also depicted, where the membrane opens.

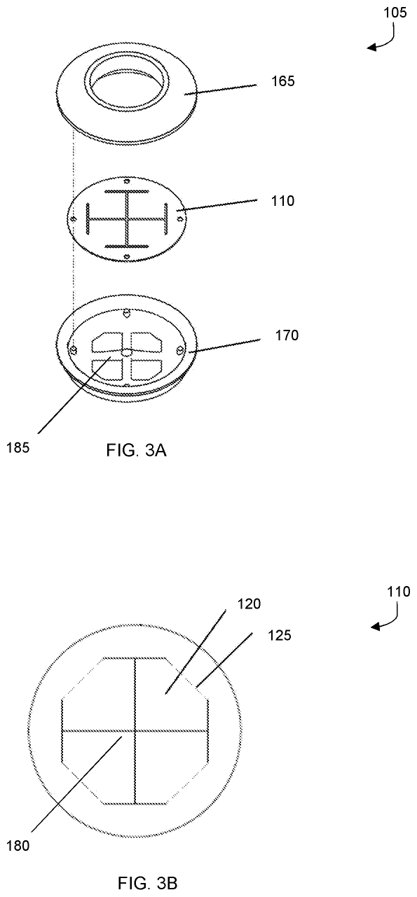

FIG. 3A depicts an exploded view of the components of a valve 105. A valve cover 165 secures the membrane 110 to a valve body 170. The membrane is a single piece of material secured around its perimeter. The membrane can be secured to the valve body at one or more specific locations around the perimeter of the membrane without compromising the function of the valve. As depicted, the membrane can be secured at four points equidistance from one another. The membrane can be secured to the valve body through various means such as one or more protrusions on the valve body closely fitting with one or more complementary holes situated in the membrane. However, those skilled in the art will appreciate other means of securing the membrane to the valve body. For example, the membrane can be secured through a single point of contact that extends around the perimeter of the membrane. In one embodiment, the membrane perimeter can be partially or entirely secured and held in place by being pinned between the valve cover and valve body, and more specifically between the valve cover and mounting surface of the valve body.

Cuts or slits in the membrane allow the formation of membrane flaps with an inner portion or free end of each flap able to flex toward the valve cover 165 and away from the valve body such that an opening is formed at the central region of the valve. The membrane permits the flow of air in one direction only. The perimeter (i.e. outer ring) of the membrane retains its structural rigidity with the cuts for the membrane flaps.

Rib portions of the valve body 185 traverse across the center of the valve body 170 and converge to form a cross shape across its central region 180. When the valve is closed, the membrane flaps are seated against these ribs to prevent air flow through the valve. The ribs 185 allow the membrane flaps to flex toward the valve cover 165 and prevent the membrane flaps from flexing in the opposite direction. This gives the valve its “unidirectional” quality as air can flow only from the valve body 170 through the valve cover 165 to the ambient environment.

The design of the multi-flap valve achieves the largest airway surface opening with the most compact valve assembly owing to the flaps facing inward toward each other and being connected to each other. This membrane design minimizes the dead space which allows more air flow to flow through the membrane while still maintaining other features such as physical integrity of the membrane and the ability of the membrane to maintain a seal while the valve is in any orientation.

FIG. 3B depicts a top view of a membrane 110 according to one embodiment. The membrane 110 is comprised of a thin flexible material that is cut into four flaps 120 (i.e. sections or panels). In this design, the membrane 110 is round and the center portion is divided into four flaps of equal size and shape. Each flap can flex independently from a hinge region 125 to open the valve at a central region 180. The length of the hinge regions can be varied (i.e. with additional or reduced cut sections) to adjust flexibility at the hinge region. The membrane is secured to a valve body 170 around its perimeter. The valve cover 165 secures the membrane to the valve body 170.

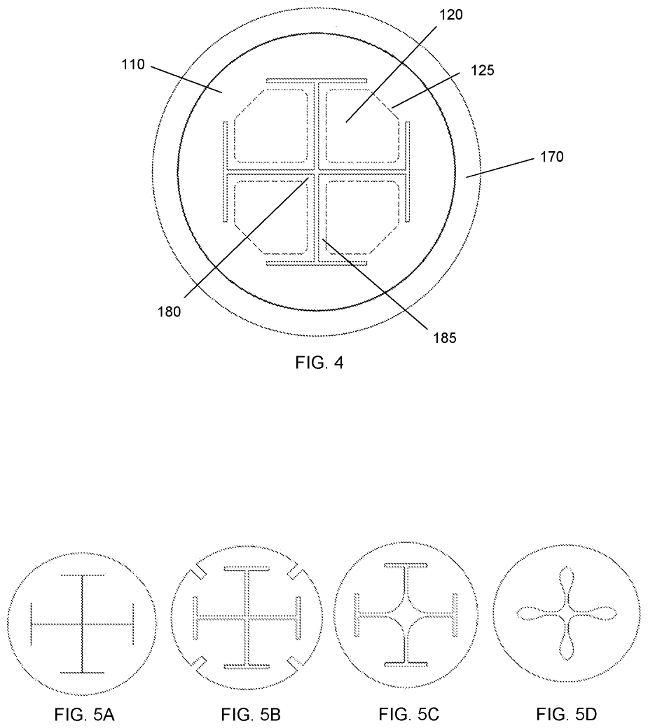

FIG. 4 depicts a top view of a membrane with areas of contact between the membrane and the valve body. A membrane gap is present between the flaps and the areas that define the hinge regions. Free edges of the membrane flaps 120 are seated over and against the sealing surface of the rib portions 185. The dashed lines represent the open areas of the valve body 170 whilst also defining the rib portions 185 of the valve body 170. As shown, the membrane gaps formed by the membrane cuts are aligned with the perimeter and rib portions 185 of the valve body 170. Air flows through the open areas when the valve is open. When the valve is closed, the membrane is sealed and tightly seated against the rib portions 185 to cover the open areas of the valve body 170.

With neutral or negative air pressure inside the mask body (during inhalation) the flaps 120 of the membrane 110 remain closed to maintain a firm seal. This prevents unfiltered air from flowing into the mask body. With positive air pressure (during exhalation) the flaps 120 flex open. This allows exhaled air to flow through the valve 105 and out of the mask body 115 with minimal resistance.

Each flap of the membrane can flex or bend to allow air from within the mask to flow through a central region or orifice and be vented out of the mask. This allows the path of the flowing exhaled air to exit through the valve perpendicular to the opening of the valve body with minimal deviation from its original trajectory. Air is met with minimal resistance because it is not diverted to peripheral edges of the membrane, but instead passes through a central region of the membrane. Accordingly, an advantage of the unidirectional valve is the ability to allow air to flow freely through the valve with minimal resistance caused by the valve flaps. As a result, more air can flow through the valve as compared to conventional valves of similar size. Moreover, the flaps maintain a firm seal to close the valve during inhalation to ensure the inhaled air is filtered through the mask material.

Each flap within the membrane is comprised of a free end at a central region 180 and a fixed end that corresponds to the hinge region 125, these ends generally being located opposite from one another. The flaps can be arranged in a radial, circular, elliptical and/or rectangular manner with the free end of each flap opening to allow air to flow through a central region, perpendicular to the valve. In one embodiment, the free end of the flaps are situated adjacent to each other. In another embodiment, there is a small gap between flaps in order to prevent overlapping of the flaps when they return to rest and seat against the sealing surface 160. The flaps open with the direction of expelled air, in a direction perpendicular to the valve body.

FIG. 5A-5D depict alternative designs of membranes. Each design can include a corresponding valve body so that each flap of the membrane forms a seal at a contact area (not shown) of the sealing surface 160 of the valve body 170. FIG. 5A depicts a membrane with four symmetrical flaps of equal size. The flaps can be formed by cutting straight lines through the membrane to form a cross-cut shape. Cuts or indentations perpendicular to the “cross-cut” shorten the length of the hinge regions.

The use of multiple flaps in the membrane is generally preferred as it can reduce resistance to airflow thus increasing the volume of air flow through the valve. For example, a membrane with four flaps presents less resistance than a membrane with a single large flap. Each of the four flaps can flex with less force than is necessary to flex a single large flap. Also, when the valve is open, the design with four flaps can yield a larger central region for the flow of air.

FIG. 5B depicts a membrane of similar design with spacing (i.e. gaps) between each flap. The spacing between the membrane flaps can prevent overlap of the flaps when they form a seal against the rib portions of the valve body. An overlap of the flaps can affect the integrity of the seal and allow unfiltered air to enter the mask body. Ridges around the perimeter can be used for aligning the membrane on the valve body.

Similarly, FIG. 5C depicts a membrane with spacing between its flaps and curved vertices or free end portions. In this design, the gap is larger in a central region. FIG. 5D depicts a membrane wherein a cutout of the center portion creates four curved flaps that are spaced apart from each other. In these examples, the flaps are of equal size and shape. However, in alternative designs, the shapes of the flaps can be different from each other. Further, the arrangement of the flaps within the membrane can be non-symmetrical. It will be appreciated that in all configurations and designs of the membrane flaps, the cut edges of each flap will seat against a rib portion of the sealing surface and cover all openings in the valve body to form a tight seal and prevent air flowing there through.

Both the design and material of the membrane can affect the flexibility of the membrane flaps and performance of the valve. A more rigid membrane material can increase the amount of force necessary to flex the flaps. Further, different lengths of the hinge region can affect the flexibility of the flaps. With a longer hinge region, more membrane material must bend which can make the hinge regions stiffer. As a result, a greater force is necessary to deflect the flap which increases the resistance to open the valve. Further, a curved hinge region can increase the stiffness of the flap.

Structural features can also be introduced into the material of the membrane (not shown) that will affect its function. For example, indentations or ribs formed in the surface of the membrane, can increase or decrease the flexibility of the flaps. A straight indentation across the hinge region will increase flexibility of the flap. In contrast, a reinforcing rib perpendicular to the hinge region can decrease the flexibility of the flap. Such structural features can be used to modify the characteristics and performance of a membrane based on user applications.

The shape and structure of the valve body can also affect membrane flexing and performance of the valve. The ratio of the perpendicular distance to the length of the base, the area moment of inertia of the beam cross-section about the axis of flexion, the elastic modulus of the material and the cantilever beam boundary condition are considered. In essence, a shorter perpendicular distance of the vertex of the flap (with respect to the length of the base and the cross-sectional area) leads to a stiffer flap. With a stiffer flap, more force is required to open the valve. Furthermore, the rigidity of the membrane can be affected by how the membrane is secured to the valve body. Securing near or at the hinge region of a flap can increase the flexibility of the flap, making it easier to open the valve.

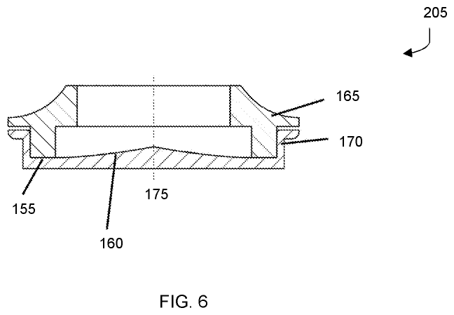

FIG. 6 depicts a cross-sectional view of a valve 205 with a curved sealing surface 160 according to one embodiment. As in FIG. 2, a valve cover 165 secures a membrane to the valve body 170. The area where the membrane is affixed or secured to valve body is known as a mounting surface 155. In one embodiment, the mounting surface 155 is flat (as shown). In another embodiment, the mounting surface 155 has a degree of curvature (biased) so that the curvature is imparted to the membrane.

When the valve is closed, each flap sits firmly atop an opening in the valve body. The flaps sit on top of the sealing surface 160 of rib portions 185 and cover the valve opening to form a seal. The sealing surface 160 can be flat or have some degree of curvature. In this example, the sealing surface 160 is higher toward the center 175. The curved surface positions the membrane away from the direction of the exhalation airflow and toward the valve cover 165 and ambient environment. This enables the membrane flaps to open easier during exhalation compared to a flat surface. The shape of the sealing surface 160 also provides support to the flaps and works in combination with the stiffness of the flaps to prevent them from collapsing inward during inhalation.

The configuration of the mounting surface 155 can also be considered along with the shape of the membrane flaps to achieve an optimal seal over the valve openings. The mounting surface 155 can be sloped at an angle with respect to the plane of the valve opening. In one embodiment, the membrane is affixed/secured to the valve body only around the perimeter or peripheral edges at one or more points. In another embodiment, the membrane is not affixed/secured to the valve body at the membrane center or central region of the membrane.

In one embodiment, the mounting surface is a single point, or a group of points, that secure the membrane to the valve. In another embodiment, the mounting surface is a line, or a set of lines, that secure the membrane in place. As presented above, the configuration of the mounting surface is dependent on the shape of the flap in order to obtain an optimal seal over the opening in the valve. The configuration of the mounting area can be a combination of some or all of the above embodiments to achieve the optimum effect (i.e. a tight seal when the valve is closed with minimal resistance to air flow to open the valve and expel air).

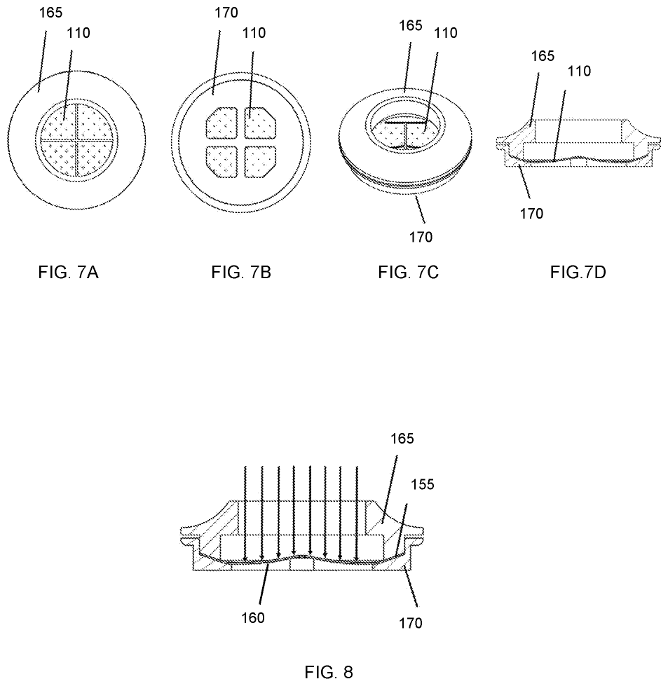

FIG. 7A-7D depict views of a valve when the membrane is closed, according to one embodiment. FIG. 7A depicts a top view which faces the outside of a mask body. A valve cover 165 secures a membrane 110 to the valve body 170. FIG. 7B depicts a bottom view of the membrane 110 and the valve body 170 which face the interior of a mask body. FIG. 7C depicts a perspective view and FIG. 7D depicts a cross-sectional view of a valve with a substantially flat sealing surface.

FIG. 8 depicts a cross-sectional view of an exhalation valve and the path of airflow during inhalation, according to one embodiment. The arrows depict air flow that occurs with negative pressure inside the mask. The membrane remains seated and firmly sealed against the sealing surface 160 of the valve body 170. Here, both the mounting surface 155 and sealing surface 160 are sloped at an angle with respect to the plane of the valve opening.

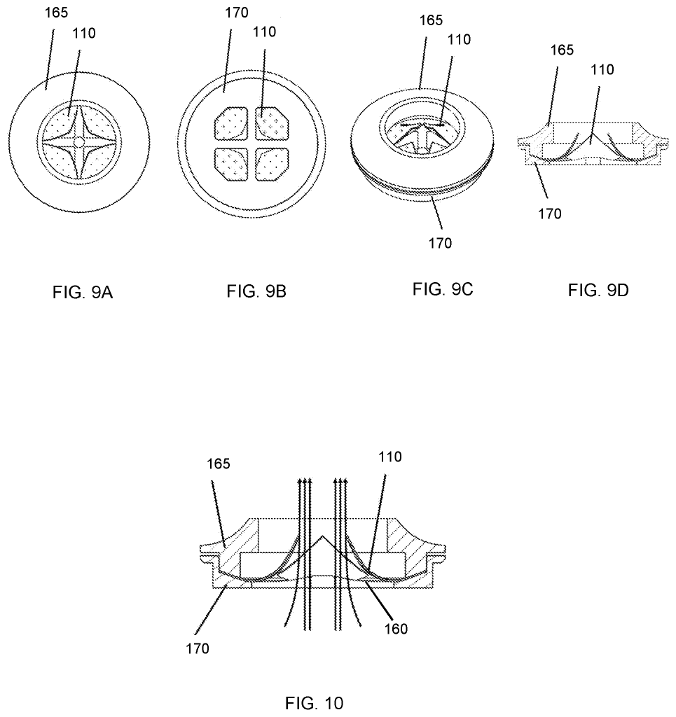

Similarly, FIG. 9A-9D depict views of a valve when the membrane is open, according to one embodiment. FIG. 9A depicts a top view which faces the exterior. FIG. 9B depicts a bottom view which faces the interior of a mask body. FIG. 9C depicts a perspective view and FIG. 9D depicts a cross-sectional view of a valve. The membrane 110 has four flaps of equal size that are arranged symmetrically with one another. As depicted, each flap flexes at a hinge region to create an opening at the center of the membrane.

FIG. 10 depicts a cross-sectional view of an exhalation valve and the path of airflow during exhalation, according to one embodiment. The arrows depict air flow from inside the mask body outward which occurs during exhalation. Positive pressure caused by exhaled airflow from inside the mask causes the flaps of the membrane to flex outward, away from the sealing surface 160. An orifice or opening of the membrane 110 is formed allowing air to flow through the valve body 170 and valve cover 165 with minimal resistance. Air flows in a direct, unobstructed path whereby the flaps do not deviate the airflow path from within the mask.

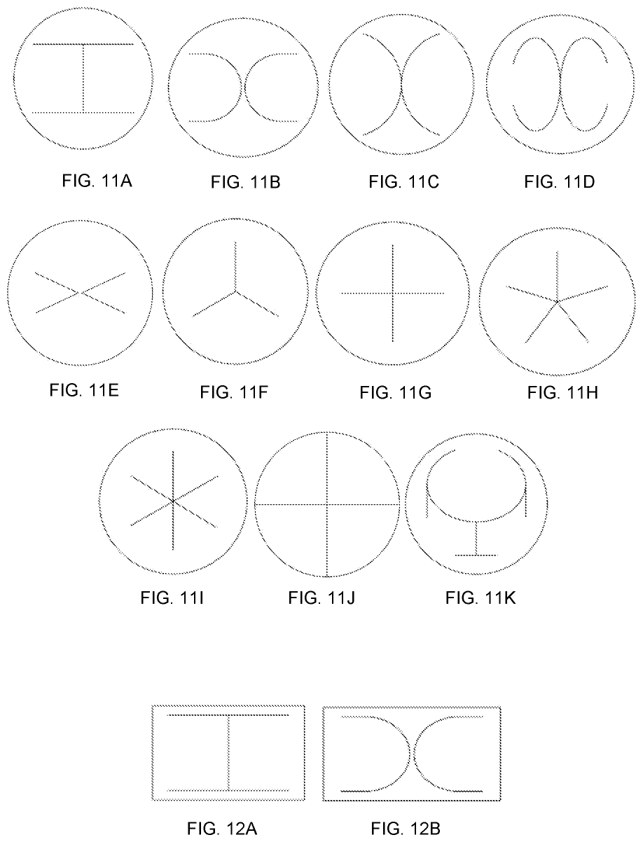

FIG. 11A-FIG. 11K depict round membranes with flaps of alternative designs, according to one embodiment. The shape of the flaps and length of the hinge regions can vary based on user needs. Each flap within the membrane is comprised of a free end and a fixed end that are located generally opposite from one another. The flaps can be arranged in a radial, circular, elliptical and/or rectangular manner with the free end of each flap opening to allow air to flow through a central region, perpendicular to the valve. Each design can include a corresponding valve body so that each flap of the membrane forms a seal at a contact area (not shown).

FIG. 11A depicts a membrane with rectangular shaped flaps. Because there are two flaps, the design is suited for a use with a valve body having a single portion (i.e. rib) that traverse across its center to form two contact areas. Similarly, FIG. 11B depicts a membrane with two curved flaps. FIG. 11C depicts a membrane with two curved flaps with a longer hinge region. FIG. 11D depicts a membrane with two elliptical flaps. FIG. 11E depicts a membrane with four triangular flaps. FIG. 11F depicts a membrane with three triangular flaps. FIG. 11G depicts a membrane with four symmetrical flaps. FIG. 11H depicts a membrane with five triangular flaps. FIG. 11I depicts a membrane with six triangular flaps. The design may be more suitable for a user seeking minimal resistance to flow through the valve. FIG. 11J depicts a membrane comprised of four panels of equal size and shape. The membrane can be comprised of individual panels rather than a single panel that is cut into portions. FIG. 11K depicts a membrane with non-identical flaps. The flaps can have different sizes and/or shapes. Here, the flaps are asymmetrical with different shapes from one another.

FIG. 12A and FIG. 12B depict membranes of rectangular shape with flaps of alterative designs, according to one embodiment. Both a rectangular valve body and rectangular valve cover would be used to secure membranes of this shape. FIG. 12A depicts a rectangular shaped membrane with two rectangular flap. FIG. 12B depicts a rectangular shaped membrane with two curved flaps.

In another embodiment, the membrane flaps, can be comprised of individual panels as depicted in FIG. 11J. The flaps or panels can be arranged in a radial, circular, elliptical or rectangular pattern with the free end of each flap opening to allow air to flow perpendicular through the valve opening. Each flap can have its own hinge region at its secured end.

An advantage of the unidirectional valve of the present invention is the ability to open asymmetrically to allow air to flow freely from any direction. This allows the valve to be placed in other parts of the facemask or respiratory device rather than placed directly in front of the nose and/or mouth. Conventional designs typically require pressure perpendicular to the valve to open the valve membrane and expel exhaled air. Thus, conventional valves may be ineffective if placed off center, away from the nose and/or mouth region. This can be restrictive to normal use of the mask and be aesthetically undesirable.

### Working Example

Use of a Facemask with a Unidirectional Valve in an Industrial Environment

A facemask creates a physical barrier between the wearer and potential contaminants in the environment. In this example, the mask body is comprised of N95 filtering material that forms a seal with the face by enclosing the nose and mouth. The mask filters at least 95% of all non-oil based airborne particles, including harmful air pollution such as PM2.5 particles, haze, volcanic ash and viruses.

Construction, manufacturing and other industrial environments can contain high levels of airborne particles such as dust and debris that pose a hazard to workers. In these environments, face masks can be essential to minimize this exposure. However, conventional masks are often unsuitable for wearing over extended periods of time.

Because air must be filtered through a porous material, conventional masks increase the resistance to breathing. More effort is required to inhale and exhale. Further, carbon dioxide, heat and moisture can accumulate inside the mask body. The masks can cause discomfort, tiredness and headaches, especially when worn over long periods of time. The unidirectional valve in the facemask can alleviate these issues.

In this example, the facemask is equipped with a unidirectional valve that is secured into the mask body. The valve body is positioned adjacent to the internal space of the mask. The valve cover faces the outside of the mask body (i.e. the ambient environment). The valve allows exhaled air to be purged from the mask body with minimal resistance and thus effort for the user.

The valve includes a membrane that is secured around its perimeter to a valve body of the unidirectional valve. The membrane is comprised of a flexible material with four flaps that move independently such that each flap flexes at a hinge region. The flaps are separated by a gap (i.e. membrane gap). The membrane opens at a central region when the flaps flex in a direction away from the valve body. This allows the path of exhaled air to exit through the valve perpendicular to the opening of the valve body with minimal deviation from its original trajectory. Air is met with minimal resistance because it is not diverted to peripheral edges of the membrane openings, but instead passes through a central region of the membrane.

The membrane can be comprised of silicone rubber or nitrile rubber, or any type of flexible elastomer or material. The membrane can have a thickness of 0.1 mm to 2 mm and a Young's Modulus between 0.001 to 0.05 GPa. In this example, the valve opening has an effective surface area of 2.68 square centimeters (cm²) but can be 2.0 cm² to 6.3 cm². Smaller valves can be ineffective as they do may provide allow sufficient airflow through the valve. Likewise, the ability of the membrane to create an effective seal can be compromised with larger valves.

A worker dons a disposable mask before entering a factory or other environment with airborne particulate matter. The body of the mask includes a unidirectional valve. The valve is secured to a side of the mask (i.e. off center). The mask can be secured with elastic bands (or similar) and the worker confirms that it fits securely to his/her head.

The unidirectional valve remains closed during inhalation. During inhalation, negative pressure inside the mask body keeps the valve closed. Individual flaps of the membrane remain seated against the valve body. The membrane remains sealed around the opening of the valve.

During exhalation, positive pressure inside the mask body opens the valve. The flaps of the membrane to flex outward (toward the exterior of the mask body). The membrane is opened allowing air to flow through the valve body with minimal resistance.

With the unidirectional valve, exhaled air is more easily expelled from the mask which reduces the buildup of heat, humidity and carbon dioxide in the mask body. This reduces the discomfort associated with wearing a conventional mask. With the unidirectional valve, the mask can be comfortably worn over extended durations of time.

It will be appreciated that variations of the above disclosed and other features and functions, or alternatives thereof, may be combined into other systems or applications. Also, various unforeseen or unanticipated alternatives, modifications, variations or improvements therein may be subsequently made by those skilled in the art which are also intended to be encompassed by the following claims.

Although embodiments of the current disclosure have been described comprehensively, in considerable detail to cover the possible aspects, those skilled in the art would recognize that other versions of the disclosure are also possible.

1.-19. (canceled)

20. A unidirectional exhalation valve for a respiratory facemask, comprising:
a) a valve body;
b) a valve cover; and
c) a membrane;
wherein the membrane is secured to the valve body around a perimeter region of the membrane by the valve cover;
wherein the membrane is comprised of a flexible material with two or more flaps that move independently such that each flap flexes at a hinge region;
wherein each of the two or more flaps comprise free peripheral edges and a free-end;
wherein the free-end of the two or more flaps are positioned adjacent to each other at the central region; and
wherein the membrane opens at a central region when the two or more flaps flex in a direction away from the valve body.

21. The unidirectional valve of claim 20, wherein the two or more flaps are formed by cuts in the membrane.

22. The unidirectional valve of claim 20, wherein the membrane forms a seal with the valve body at a sealing surface, and
wherein the sealing surface has a substantially flat shape.

23. The unidirectional valve of claim 20, wherein the membrane forms a seal with the valve body at a sealing surface, and
wherein the sealing surface has a curved shape.

24. The unidirectional valve of claim 20, wherein the membrane is secured to the valve body at a mounting surface, and
wherein the mounting surface has a substantially flat shape.

25. The unidirectional valve of claim 20, wherein the membrane is secured to the valve body at a mounting surface, and
wherein the mounting surface has a curved shape.

26. The unidirectional valve of claim 24, wherein the mounting surface is comprised of one or more points where the membrane is affixed to the valve body and/or the valve cover.

27. The unidirectional valve of claim 20, wherein the two or more flaps are formed from curved vertices in the membrane.

28. The unidirectional valve of claim 20, wherein the flaps are individual panels.

29. The unidirectional valve of claim 20, wherein the valve body comprises rib portions, and wherein the free peripheral edges of the two or more flaps are seated over and against the surface of the rib portions.

30. A membrane for a unidirectional exhalation valve in a respiratory facemask, said membrane comprised of:
a flexible material with two or more flaps that move independently such that each flap flexes at a hinge region;
wherein each of the two or more flaps comprise free peripheral edges and a free-end,
wherein the membrane is secured around a perimeter region to a valve body of the unidirectional valve;
wherein the membrane opens at a central region when the two or more flaps flex in a direction away from the valve body, and
wherein the free-end of the two or more flaps are positioned adjacent to each other at the central region.

31. The membrane of claim 30, wherein the two or more flaps are formed by symmetrical cuts that form a cross-shape in the membrane.

32. The membrane of claim 30, wherein the membrane forms a seal with the valve body at a flat sealing surface.

33. The membrane of claim 30, wherein the membrane forms a seal with the valve body at a curved sealing surface.

34. The membrane of claim 30, wherein the two or more flaps are comprised of individual panels.

35. The membrane of claim 30, wherein the two or more flaps are formed from curved vertices in the membrane.

36. The membrane of claim 30, wherein the membrane is secured to the valve body at a mounting surface, and
wherein the mounting surface has a substantially flat shape.

37. The membrane of claim 30, wherein the membrane is secured to the valve body at a mounting surface, and
wherein the mounting surface has a curved shape.

38. The membrane of claim 36, wherein the mounting surface is comprised of one or more points where the membrane is affixed to the valve body and/or a valve cover.

39. The membrane of claim 30, wherein the valve body comprises rib portions, and wherein the free peripheral edges of the two or more flaps are seated over and against the surface of the rib portions.
